(19) United States
(12) Patent Application Publication
(10) Pub. No.: US 2017/0150940 A1
(43) Pub. Date: Jun. 1, 2017

(54) AIR CALIBRATION

(71) Applicant: Shenyang Neusoft Medical Systems Co., Ltd., Shenyang (CN)

(72) Inventors: Shanshan LOU, Shenyang (CN); Jiaxiang XIE, Shenyang (CN)

(21) Appl. No.: 15/232,546

(22) Filed: Aug. 9, 2016

(30) Foreign Application Priority Data
Dec. 1, 2015 (CN) 201510866804.4

(51) Int. Cl. A61B 6/00; A61B 6/06; A61B 6/03

(52) U.S. Cl. CPC A61B 6/582; A61B 6/032; A61B 6/06

(57) ABSTRACT

The present disclosure includes: in a collimation condition, performing air scan based on a predetermined reference scanning condition to obtain air calibration data corresponded to the predetermined reference scanning condition; and obtaining air calibration data of non-reference scanning conditions in the collimation condition based on the air calibration data corresponded to the predetermined reference scanning condition and stored incremental differences, wherein the stored incremental differences are differences between the air calibration data of the predetermined reference scanning condition and the non-reference scanning conditions.

### BACKGROUND

The present disclosure relates to air calibration of a medical imaging device.

Medical imaging apparatus has become a routine diagnostic technique in hospital. As using the medical imaging apparatus, i.e., the Computed Tomography (CT) apparatus, the Positron Emission Tomography (PET) apparatus and other medical imaging apparatus, it may perform a plurality of calibrations before image scanning to increase the accuracy of the scanning data for improving the image quality. Air calibration is one of the calibrations. By performing the air calibration, it may obtain the air calibration data in a specific scanning condition. Thus, in the specific scanning condition, it may subtract the air calibration data from the real scanned data to increase the accuracy of the scanned data.

NEUSOFT MEDICAL SYSTEMS CO., LTD. (NMS), founded in 1998 with its world headquarters in China, is a leading supplier of medical equipment, medical IT solutions, and healthcare services. NMS supplies medical equipment with a wide portfolio, including CT, Magnetic Resonance Imaging (MRI), digital X-ray machine, ultrasound, Positron Emission Tomography (PET), Linear Accelerator (LINAC), and biochemistry analyser. Currently, NMS' products are exported to over 60 countries and regions around the globe, serving more than 5,000 renowned customers. NMS's latest successful developments, such as 128 Multi-Slice CT Scanner System, Superconducting MRI, LINAC, and PET products, have led China to become a global high-end medical equipment producer. As an integrated supplier with extensive experience in large medical equipment, NMS has been committed to the study of avoiding secondary potential harm caused by excessive X-ray irradiation to the subject during the CT scanning process.

### BRIEF DESCRIPTION OF DRAWINGS

Features of the present disclosure are illustrated by way of example and not limited in the following figure(s), in which like numerals indicate like elements, in which:

FIG. 1 is a flowchart of air calibration according to an example of the present disclosure;

FIG. 2 is a schematic view of two air calibration curves corresponding to scanning condition 1 and scanning condition 2 according to the present disclosure;

FIG. 3 is a schematic view of incremental differences between two air calibration curves in FIG. 2 according to the present disclosure;

FIG. 4 is a flowchart of method for obtaining incremental differences according to an example of the present disclosure;

FIG. 5 is a schematic diagram of a system structure of a medical imaging apparatus according to the present disclosure; and

FIG. 6 is a schematic diagram of function modules of air calibration control logic for air calibration according to an example of the present disclosure.

### DETAILED DESCRIPTION

For simplicity and illustrative purposes, the present disclosure is described by referring mainly to an example thereof. In the following description, numerous specific details are set forth in order to provide a thorough understanding of the present disclosure. It will be readily apparent however, that the present disclosure may be practiced without limitation to these specific details. In other instances, some methods and structures have not been described in detail so as not to unnecessarily obscure the present disclosure. As used herein, the terms “a” and “an” are intended to denote at least one of a particular element, the term “includes” means includes but not limited to, the term “including” means including but not limited to, and the term “based on” means based at least in part on.

Before using the medical imaging apparatus, such as the CT apparatus, to scan the patient, air calibration is one of the calibration processes of the medical imaging apparatus. By performing the air calibration, it may obtain the air calibration data corresponding to a specific scanning condition. It may subtract the air calibration data from the real scanned data of the patient to increase the accuracy of the scanned data and further increase the quality of the scanned image.

In an example of the present disclosure, the air calibration of the medical imaging apparatus, such as the CT apparatus, may be divided into a first type calibration and a second type calibration. When the medical imaging apparatus is shipped out from the factory, it may be performed the first type calibration. The medical imaging apparatus may be performed the first type calibration in a long time interval. The second type calibration may be performed for routine maintenance. Owing to the influence of ambient temperature and humidity, the medical imaging apparatus may be performed the second type calibration for regular routine maintenance. For example, the second type calibration may be performed in every seven days. Thus, the second type calibration may be performed with high frequency.

In performing the second type calibration each time, such as the air calibration, it usually obtains the air calibration data corresponding to all the pre-determined scanning conditions. For example, “A tube voltage is 80 KV, a rotation speed is 0.6 s, and a small focus” is a scanning condition. The CT apparatus may perform air scan according to the scanning condition to obtain the scanned data corresponding to the scanning condition. “A tube voltage is 80 KV, a rotation speed is 0.6 s, and a large focus” is another scanning condition. The CT apparatus may perform air scan according to another scanning condition to obtain the scanned data corresponding to another scanning condition. In general, the number of scanning conditions which are used to perform the air calibration may be very large.

The pre-determined scanning conditions may include all the scanning conditions of each collimation condition. The scanning conditions may comprises a tube voltage, a rotation speed, a size of focus, a position of focus, and a position of a filter, wherein the position of a filter may refer to the specific location of a shielding apparatus in the X-ray light path. It may use the tube voltage, the rotation speed, and the size of focus to describe the scanning condition. It is assumed that the scan parameters A, B, and C may respectively refer to the tube voltage, the rotation speed, and the size of focus, and the selection number of the scan parameters A, B, and C may be m, n, and p, respectively. That is, the mathematical expressions of the scan parameters A, B, and C may be shown as following:

Ai: i=1 to m,

Bj: j=1 to n, and

Ck: k=1 top.

Therefore, the number of the scanning conditions may be T (=m*n*p). That is, there is T scanning conditions for each collimation condition. When there are x collimation conditions, the total number of the scanning conditions may be x*T. For example, it is assumed that the number of scan parameter A corresponding to the tube voltage is equal to 4, i.e., m=4, the number of scan parameter B corresponding to the rotation speed is equal to 2, i.e., n=2, and the number of scan parameter C corresponding to the size of focus is equal to 2, i.e., p=2, then, T is equal to 16. When there are three collimation conditions, the total number of the scanning conditions may be equal to 48. In each of the collimation conditions, it may perform air scans to obtain the air calibration data for all the scanning condition.

According to the conventional scan method, in performing the second type calibration, it may perform air scans for T scanning conditions in each collimation condition, resulting in time consuming of the air calibration and low efficiency. The present disclosure provides an air calibration method to minimize the number of executions of the second type calibration, thereby improving the efficiency of air calibration.

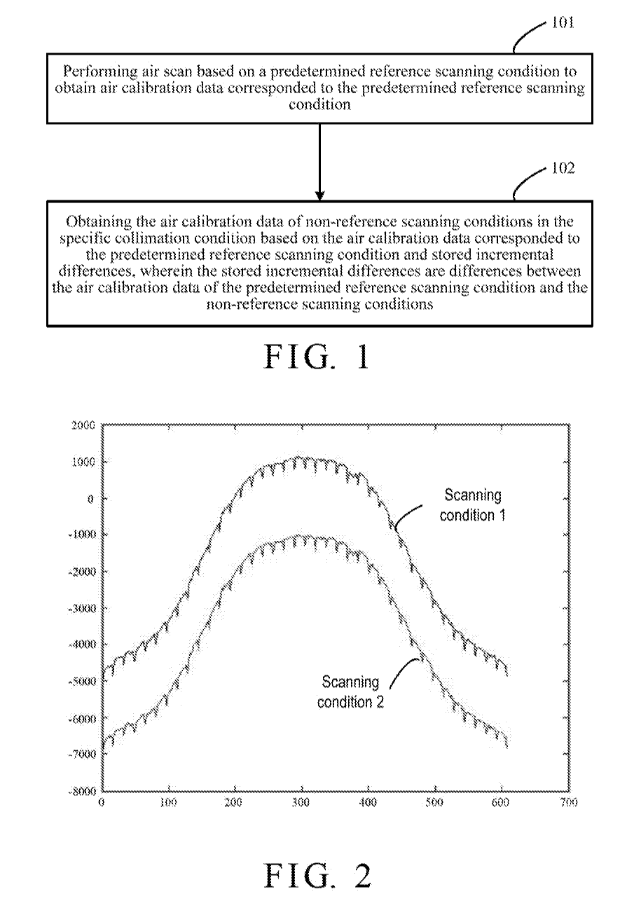

In the example of air calibration of the present disclosure, in the execution of the second type calibration, it is based on the practical experience that “Under the same collimation condition, the incremental differences of the air calibration data corresponding to different scanning conditions are fixed.” According to the practical experience, it may simplify the air calibration method in the present disclosure. That is, it may perform air scans for part of scanning conditions to obtain the corresponding air calibration data. The air calibration data corresponding to the other scanning conditions may be obtained by calculation according to the incremental differences. The scanning conditions which are used to perform air scan may be classified as the first type of scanning condition. The other scanning conditions which are obtained by calculating with incremental differences may be classified as the second type of scanning conditions. FIG. 1 is a flowchart of air calibration according to an example of the present disclosure. As shown in FIG.1, it includes block 101 and block 102.

In block 101, in a specific collimation condition, it may perform air scan based on a predetermined reference scanning condition to obtain air calibration data corresponded to the predetermined reference scanning condition.

In block 102, it may obtain the air calibration data of non-reference scanning conditions in the specific collimation condition based on the air calibration data corresponded to the predetermined reference scanning condition and stored incremental differences, wherein the stored incremental differences are differences between the air calibration data of the predetermined reference scanning condition and the non-reference scanning conditions.

In an example of the present disclosure, the scanning condition may include a tube voltage, a rotation speed, and a size of focus. It is assumed that the number of the tube voltage is equal to 4, the number of the rotation speed is equal to 2, and the number of the size of focus is equal to 2, then there may have 16 scanning conditions in the specific collimation condition. In block 101, it may use the “Tube voltage is 120 KV, the rotational speed is 1 s, and small focus” condition as the reference scanning condition

In the block 102, the scanning conditions other than the reference scanning condition may be regarded as the non-reference scanning conditions. The air calibration data corresponding to the non-reference scanning conditions may be obtained without performing the air scan based on the non-reference scanning conditions. That is, air calibration data corresponding to the non-reference scanning conditions may be obtained according to the air calibration data corresponded to the reference scanning condition and pre-stored incremental differences.

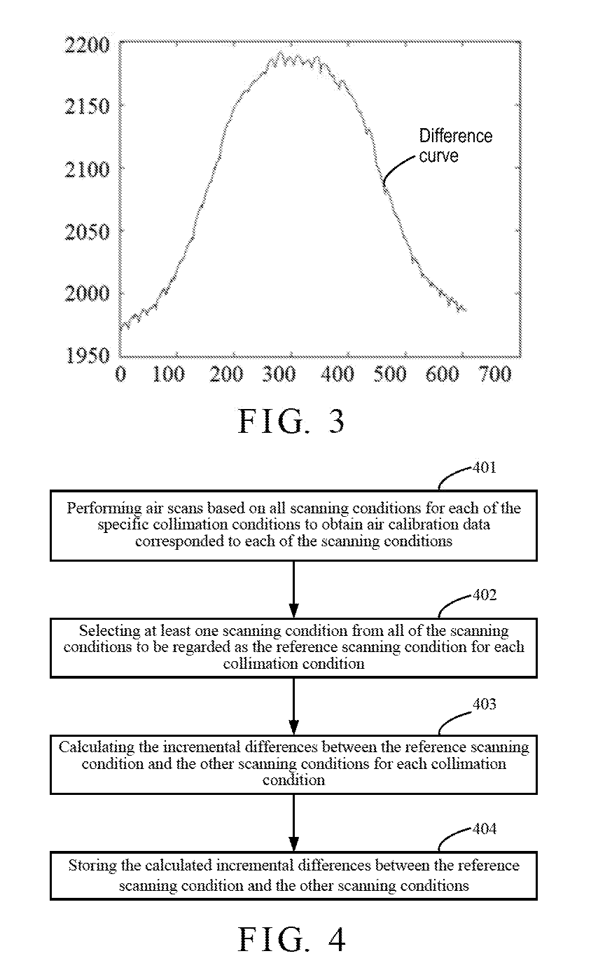

For example, it may be pre-stored the incremental differences between the air calibration data of the reference scanning condition and the non-reference scanning conditions. FIG. 2 is a schematic view of two air calibration curves corresponding to scanning condition 1 and scanning condition 2 according to the present disclosure. FIG. 3 is a schematic view of incremental differences between two air calibration curves in FIG. 2 according to the present disclosure. As shown in FIG. 2, the scanning condition 1 may have scan parameters “Tube voltage is 120 KV, the rotational speed is 1 s, and small focus”, and the scanning condition 2 may have scan parameters “Tube voltage is 140 KV, the rotational speed is 1 s, and small focus”. When the medical imaging apparatus performs air scan according to the scanning condition 1 and the scanning condition 2, it may obtain two air calibration curves as shown in FIG. 2. The data corresponding to the two air calibration curves is regarded as the air calibration data. The difference curve is the differences between the two air calibration curves and is shown in FIG. 3. As shown in FIG. 3, the difference curve is the incremental differences which may represent the differences between the air calibration data of the reference scanning condition and the non-reference scanning conditions. Therefore, if the incremental differences between two scanning conditions and the air calibration curve of one scanning condition are known, then the air calibration curve of the other scanning condition may be obtained.

Based on the above principles, it may be pre-stored the incremental differences between the reference scanning condition and the non-reference scanning conditions. An incremental difference is the difference between the air calibration data on the reference scanning condition and the air calibration data on the other scanning condition. Thus, there will be a plurality of incremental differences which may be grouped as an incremental difference set. After obtaining the air calibration data corresponded to the reference scanning condition in block 101, it may calculate and obtain the air calibration data of the other scanning conditions according to the incremental difference. The other scanning conditions may be the scanning conditions which have not been performed the air scan in the same time for the same specific collimation condition.

FIG. 4 is a flowchart of method for obtaining incremental differences according to an example of the present disclosure.

In block 401, it may perform air scans based on all scanning conditions for each of the specific collimation conditions to obtain air calibration data corresponded to each of the scanning conditions.

For example, the operations in block 401 may be performed in the first type calibration, that is, the operations in block 401 may be performed before the medical imaging apparatus, such as CT apparatus, being shipped out from the factory. It may perform air calibration for all of the scanning conditions before the medical imaging apparatus being shipped out from the factory to obtain the air calibration data corresponding to all of the scanning conditions. Alternatively, after a long time interval without using the medical imaging apparatus, it may perform air calibration for all of the scanning conditions before re-starting the medical imaging apparatus. For example, after a few months without using the medical imaging apparatus, it may obtain the air calibration data corresponding to all of the scanning conditions.

In block 402, it may select at least one scanning condition from all of the scanning conditions to be regarded as the reference scanning condition for each collimation condition.

For example, for each collimation condition, it may select at least one scanning condition which may be regarded as the reference scanning condition. Alternatively, the reference scanning conditions may be the same or different for all of the collimation conditions. For example, the reference scanning condition may be “Tube voltage is 120 KV, the rotational speed is 1 s, and small focus” for all collimation conditions.

For the selection of the reference scanning conditions, it may choose any one, or it may choose a reference scanning condition with higher clinical utilization rate. For example, in the aforementioned 16 scanning conditions, some scanning conditions may be had relatively higher clinical utilization rate, and some scanning conditions may be seldom used. It may choose the scanning conditions with relatively high frequency utilization rate as the reference scanning conditions. The utilization rate may be ranked according to the clinical usage rate, for example, the clinical usage rate of a scanning condition may be the number of the scanning condition which had been used to perform air scan within a predetermined time interval. It may choose the first ranked scanning condition as the reference scanning condition.

In block 403, it may calculate the incremental differences between the reference scanning condition and the other scanning conditions for each collimation condition.

For example, under each of the collimation conditions, it may choose the reference scanning condition for each collimation condition. Therefore, in block 403, for each collimation condition, it may calculate the incremental differences between the reference scanning condition and the other scanning conditions in the collimation condition.

From the actual experience, it may be shown that the incremental differences of the air calibration data corresponding to different scanning conditions are fixed under the same collimation condition. That is, after obtaining the incremental differences between the reference scanning condition and the other scanning conditions in a specific collimation condition, in the air re-scanning operation of the CT apparatus for the routine maintenance, the scanned air calibration data corresponding to different scanning conditions are variable. But, the incremental differences of the air calibration data corresponding to different scanning conditions are fixed under the same collimation condition. Therefore, the incremental differences between the reference scanning condition and the other scanning conditions may be relatively fixed under the same collimation condition.

In block 404, it may store the calculated incremental differences between the reference scanning condition and the other scanning conditions.

It may store the incremental differences between the reference scanning condition and the other scanning conditions, wherein the incremental differences are calculated in block 403. Thus, as performing the subsequently daily air calibration, it may perform the air scan according to the reference scanning condition, obtain the air calibration data corresponding to the reference scanning condition, and then obtain the air calibration data of the other scanning conditions based on the stored incremental differences. Thus, it may obtain the air calibration data of the other scanning conditions without performing air scan. It may be dramatically reduced the number of air scan in the air calibration so as to simplify the process and increase the efficiency of air calibration.

Taking the scanning conditions of the collimation condition for example, it may select one reference scanning condition in the collimation condition. Alternatively, it may still perform air scan to obtain the air calibration data for the scanning conditions other than the reference scanning condition. That is, the number of the scanning conditions, which are used to perform air scan to obtain the air calibration data corresponded, may be more than one, for example, may be two, three, four and so on. The scanning conditions, which are used to perform air scan to obtain the air calibration data corresponded, may be regarded as the performed scanning conditions. The scanning conditions, which are not used to perform air scan to obtain the air calibration data, may be regarded as the non-performed scanning conditions. According to an example of the present disclosure, the performed scanning conditions may be a plurality of the scanning conditions which also include the reference scanning condition. For another example of the present disclosure, the performed scanning conditions may only include the reference scanning condition, or the reference scanning condition and several other non-reference scanning conditions. Either way, for all the scanning conditions of the specific collimation condition, it may perform air scan according to part of all scanning conditions rather than to perform air scan according to all scanning conditions. Therefore, it may dramatically reduce the number of air scans. For the non-performed scanning conditions, the air calibration data may be obtained according the air calibration data corresponded to the reference scanning condition and pre-stored incremental differences.

Alternatively, taking the scanning conditions of the collimation condition for example, it may select more than one reference scanning conditions for the collimation condition. For example, it may select two reference scanning conditions for the collimation condition. It may be calculated and pre-stored the incremental differences between the air calibration data of the two reference scanning conditions and the non-reference scanning conditions. Thus, as performing the subsequently routine maintenance, it may select one reference scanning condition from the at least two reference scanning conditions to perform the air scan, and calculate to obtain the air calibration data corresponded to the other non-reference scanning conditions according to the incremental differences between the selected reference scanning condition and the other non-reference scanning conditions.

Further, when the number of the reference scanning conditions is more than one, it may also calculate the air calibration data corresponding to the non-reference scanning conditions according to the plurality of the reference scanning conditions, in which the calculation is not necessarily based on only one reference scanning conditions. In the specific implementation, for the non-reference scanning condition, it may calculate the air calibration data based on the incremental differences between the the non-reference scanning condition and the reference scanning condition, in which the reference scanning condition is closest to the non-reference scanning condition. The closest reference scanning condition may have maximum common scan parameters with the non-reference scanning condition. For example, it is assumed that the first reference scanning condition may have scan parameters “Tube voltage is 120 KV, the rotational speed is 1 s, and small focus”, and the second reference scanning condition may have scan parameters “Tube voltage is 120 KV, the rotational speed is 0.6 s, and small focus”. Then, for the non-reference scanning condition with scan parameter of the rotational speed being 0.6, it may calculate the air calibration data according to the incremental differences between the non-reference scanning condition and the second reference scanning condition. For the non-reference scanning condition with scan parameter of the rotational speed being 1, it may calculate the air calibration data according to the incremental differences between the non-reference scanning condition and the first reference scanning condition. In this way, it may further improve the accuracy of the air calibration data.

For an example of air calibration in the present disclosure, it may obtain the air calibration data of the other scanning conditions by the pre-stored incremental differences and the air calibration data of the reference scanning condition. It may perform air scan for part of scanning conditions rather than to perform air scan for all scanning conditions. It may be dramatically reduced the number of air scan in the air calibration so as to simplify the process and increase the efficiency of air calibration.

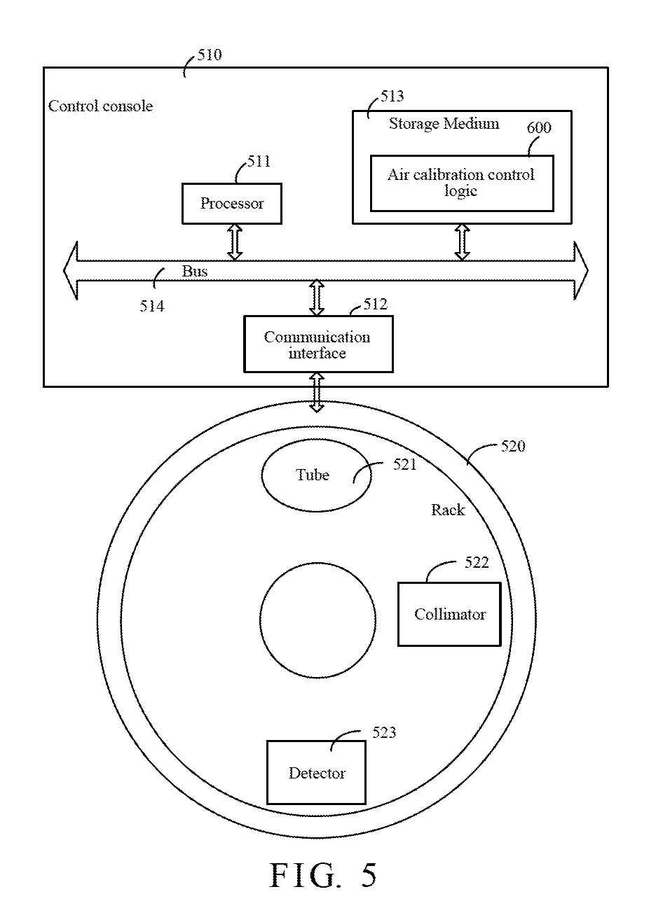

The aforementioned air calibration method may be performed in a medical imaging apparatus, such as the CT apparatus. FIG. 5 is a schematic diagram of a system structure of a medical imaging apparatus according to the present disclosure. As shown in FIG. 5, the medical imaging apparatus may include a control console 510 and a rack 520. The control console 510 may include a processor 511, communication interface 512, storage medium 513, and a bus 514. The processor 511, communication interface 512, and storage medium 513 may be communicated to each other through the bus. The rack 520 may include a tube 521 of X-ray source, a collimator 522, and a detector 523.

The storage medium 513 may be stored the air calibration control logic 600 corresponding to the machine-executable instructions executable by the processor 511. The storage medium 513 in which the machine readable instructions are stored may be a non-volatile memory or storage media including by way of example semiconductor memory devices, e.g., EPROM, EEPROM, DRAM and flash memory devices; magnetic disks, e.g., internal hard disks or removable disks; magneto optical disks; and CD ROM and DVD-ROM disks. The processor 511 may read the instructions of the corresponding modules of the air calibration control logic 600 stored in the storage medium 513 and executes the instructions to perform the aforementioned air calibration method. For example, the machine-executable instructions, corresponding to the air calibration control logic 600, may be the corresponding function procedure of the software control program of the computer. During executing the instructions by the processor 510, the control console may display a function interface corresponding to the instructions in a display interface.

If the machine-executable instructions, corresponding to the air calibration control logic 600, are implemented as software functions, and are sold or used as an independent product, then the machine-executable instructions may be stored in a storage which can be read by a computer or a processor. Based on this understanding, the essence of the technical solution of the present disclosure, part of the technical solution of the present disclosure with contribution for the prior art or part of the technical solution of the present disclosure may be in a form of a software product. The software product may be stored in a storage media which comprises a plurality of instructions that when executed by a computer cause the computer to perform all the methods or part of methods disclosed in the present disclosure, wherein the computer may be a personal computer, server, or a network apparatus and so on. The storage media may be a U-disk, a removable hard disk, a read only memory (ROM), a random access memory (RAM), a floppy disk or a CD-ROM in which may be stored program codes.

As performing the air calibration, the processor 511 may read the machine-executable instructions corresponding to the air calibration control logic 600. The instructions executed by the processor 511 may cause the processor 511 to perform the following operations.

The processor 511 may perform air scans based on a predetermined reference scanning condition under a specific collimation conditions to obtain air calibration data corresponded to the predetermined reference scanning condition.

The processor 511 may calculate and obtain air calibration data of non-reference scanning conditions in the specific collimation condition based on the air calibration data corresponded to the predetermined reference scanning condition and stored incremental differences, wherein the stored incremental differences are differences between the air calibration data of the predetermined reference scanning condition and the non-reference scanning conditions.

According to an example, before performing air scan based on the predetermined reference scanning condition to obtain air calibration data corresponded to the predetermined reference scanning condition, the machine readable instructions are further to cause the processor 511 to perform the following operations.

The processor 511 may perform air scans based on all pre-determined scanning conditions in the specific collimation condition to obtain air calibration data corresponded to each of the scanning conditions, wherein each of the scanning conditions may comprise at last one scan parameter of the following: a tube voltage, a rotation speed, a size of focus, a position of focus and a position of a filter.

The processor 511 may select at least one scanning condition from all of the scanning conditions to be regarded as the reference scanning condition.

The processor 511 may calculate and store the incremental differences between the reference scanning condition and the other reference scanning conditions in the all pre-determined scanning conditions, wherein the stored incremental differences are differences between the air calibration data of the reference scanning condition and the other scanning conditions.

Further, in the selection of at least one reference scanning condition from all of the scanning conditions being the reference scanning condition, the machine readable instructions are further to cause the processor 511 to perform the following operations.

The processor 511 may select the at least one reference scanning condition from a pre-determined ranking range of clinical usage of all scanning conditions to be the reference scanning condition, wherein the pre-determined ranking range is first N scanning conditions which are sorted according to the clinical usage from highest ranking to lowest ranking in all of the scanning conditions, where N is an integer greater than or equal to 1.

According to an example, in the case of multiple reference scanning conditions, the calculation of air calibration data of non-reference scanning conditions in the specific collimation condition based on the air calibration data corresponded to the predetermined reference scanning conditions and the stored incremental differences, the machine readable instructions are to cause the processor 511 to perform the following operations.

The processor 511 may select one reference scanning condition from the multiple reference scanning conditions, wherein the selected reference scanning condition is closest to the non-reference scanning condition.

The processor 511 may calculate the air calibration data of non-reference scanning conditions in the specific collimation condition based on the air calibration data corresponded to the selected reference scanning condition.

Further, in the selection of one reference scanning condition which is closest to the non-reference scanning condition from the multiple reference scanning conditions, the machine readable instructions are to cause the processor 511 to perform the following operations. The processor 511 may select one reference scanning condition from the multiple reference scanning conditions, wherein coincidence between the scan parameters of the selected reference scanning condition and the scan parameters of the non-reference scanning condition is highest.

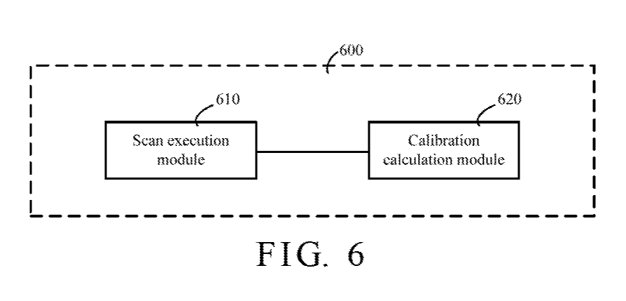

From the division of functionality, the aforementioned air calibration control logic 600 may be divided into a plurality of function modules. The specific works of each module may be combined with the aforementioned methods. FIG. 6 is a schematic diagram of function modules of air calibration control logic 600 for air calibration according to an example of the present disclosure. As shown in FIG. 6, the air calibration control logic 600 may be divided into a scan execution module 610 and a calibration calculation module 620.

In a specific collimation condition, the scan execution module 610 may perform air scan based on a predetermined reference scanning condition to obtain air calibration data corresponded to the predetermined reference scanning condition.

The calibration calculation module 620 may calculate air calibration data of non-reference scanning conditions in the specific collimation condition based on the air calibration data corresponded to the predetermined reference scanning condition and stored incremental differences, wherein the stored incremental differences are differences between the air calibration data of the predetermined reference scanning condition and the non-reference scanning conditions.

Further, before performing air scan based on the predetermined reference scanning condition to obtain air calibration data corresponded to the predetermined reference scanning condition, the scan execution module 610 may perform air scans based on all pre-determined scanning conditions in the specific collimation condition to obtain air calibration data corresponded to each of the scanning conditions, select at least one scanning condition from all of the scanning conditions to be regarded as the reference scanning condition, and calculate and store the incremental differences between the reference scanning condition and the other reference scanning conditions in the all pre-determined scanning conditions, wherein the stored incremental differences are differences between the air calibration data of the reference scanning condition and the other scanning conditions, wherein each of the scanning conditions may comprise at last one scan parameter of the following: a tube voltage, a rotation speed, a size of focus, a position of focus and a position of a filter.

Further, the selected reference scanning condition may be in a pre-determined ranking range of clinical usage, wherein the pre-determined ranking range is first N scanning conditions which are sorted according to the clinical usage from highest ranking to lowest ranking in all of the scanning conditions, where N is an integer greater than or equal to 1.

Further, in the case of multiple reference scanning conditions, the calibration calculation module 620 may select one reference scanning condition from the multiple reference scanning conditions, wherein the selected reference scanning condition is closest to the non-reference scanning condition. Then, the calibration calculation module 620 may calculate the air calibration data of non-reference scanning conditions in the specific collimation condition based on the air calibration data corresponded to the selected reference scanning condition and the pre-stored incremental differences. Wherein, in the selection of one reference scanning condition which is closest to the non-reference scanning condition from the multiple reference scanning conditions, the calibration calculation module 620 may select one reference scanning condition from the multiple reference scanning conditions, wherein coincidence between the scan parameters of the selected reference scanning condition and the scan parameters of the non-reference scanning condition is highest.

Further, in the case of multiple reference scanning conditions, the calibration calculation module 620 may select one reference scanning condition from the multiple reference scanning conditions, wherein the selected reference scanning condition is closest to the non-reference scanning condition, and calculate the air calibration data of non-reference scanning conditions in the specific collimation condition based on the air calibration data corresponded to the selected reference scanning condition. Wherein coincidence between the scan parameters of the selected reference scanning condition and the scan parameters of the non-reference scanning condition is highest.

The above are only preferred examples of the present disclosure is not intended to limit the disclosure within the spirit and principles of the present disclosure, any changes made, equivalent replacement, or improvement in the protection of the present disclosure should contain within the range.

The methods, processes and units described herein may be implemented by hardware (including hardware logic circuitry), software or firmware or a combination thereof. The term ‘processor’ is to be interpreted broadly to include a processing unit, ASIC, logic unit, or programmable gate array etc. The processes, methods and functional units may all be performed by the one or more processors; reference in this disclosure or the claims to a ‘processor’ should thus be interpreted to mean ‘one or more processors’.

Further, the processes, methods and functional units described in this disclosure may be implemented in the form of a computer software product. The computer software product is stored in a storage medium and comprises a plurality of instructions for making a processor to implement the methods recited in the examples of the present disclosure.

The figures are only illustrations of an example, wherein the units or procedure shown in the figures are not necessarily essential for implementing the present disclosure. Those skilled in the art will understand that the units in the device in the example can be arranged in the device in the examples as described, or can be alternatively located in one or more devices different from that in the examples. The units in the examples described can be combined into one module or further divided into a plurality of sub-units.

Although the flowcharts described show a specific order of execution, the order of execution may differ from that which is depicted.

For example, the order of execution of two or more blocks may be changed relative to the order shown. Also, two or more blocks shown in succession may be executed concurrently or with partial concurrence. All such variations are within the scope of the present disclosure.

Throughout the present disclosure, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An air calibration method, comprises:
in a collimation condition, performing air scan based on a predetermined reference scanning condition to obtain air calibration data corresponded to the predetermined reference scanning condition; and
obtaining air calibration data of non-reference scanning conditions in said collimation condition based on the air calibration data corresponded to the predetermined reference scanning condition and stored incremental differences, wherein said incremental differences stored are differences between the air calibration data of the non-reference scanning conditions and the predetermined reference scanning condition.

2. The air calibration method of claim 1, before performing air scan based on the predetermined reference scanning condition to obtain air calibration data corresponded to the predetermined reference scanning condition, the method further comprises:
performing air scans based on all pre-determined scanning conditions in the collimation condition to obtain air calibration data corresponded to each of the scanning conditions;
selecting at least one scanning condition from all of the scanning conditions to be regarded as the reference scanning condition; and
calculating and storing the incremental differences between the reference scanning condition and the other scanning conditions in the all pre-determined scanning conditions, wherein the stored incremental differences are differences between the air calibration data of the reference scanning condition and the other scanning conditions.

3. The air calibration method as claimed in claim 2, wherein each of the scanning conditions comprises at last one scan parameter of the following: a tube voltage, a rotation speed, a size of focus, a position of focus, and a position of a filter.

4. The air calibration method as claimed in claim 2, wherein the selection of at least one reference scanning condition from all of the scanning conditions being the reference scanning condition, the method further comprises:
selecting at least one reference scanning condition from a pre-determined ranking range of clinical usage of all scanning conditions to be the reference scanning condition.

5. The air calibration method as claimed in claim 4, wherein the pre-determined ranking range is first N scanning conditions which are sorted according to the clinical usage from highest ranking to lowest ranking in all of the scanning conditions, where N is an integer greater than or equal to 1.

6. The air calibration method as claimed in claim 1, in the case of multiple reference scanning conditions, the obtaining air calibration data of non-reference scanning conditions in the collimation condition based on the air calibration data corresponded to the predetermined reference scanning conditions and the stored incremental differences comprises:
selecting one reference scanning condition from the multiple reference scanning conditions, wherein the selected reference scanning condition is closest to the non-reference scanning condition; and
obtaining the air calibration data of non-reference scanning condition in the collimation condition based on the air calibration data corresponded to the selected reference scanning condition.

7. The air calibration method as claimed in claim 6, wherein the selection of one reference scanning condition which is closest to the non-reference scanning condition from the multiple reference scanning conditions comprises;
selecting one reference scanning condition from the multiple reference scanning conditions, wherein coincidence between the scan parameters of the selected reference scanning condition and the scan parameters of the non-reference scanning condition is highest.

8. A device for air calibration comprising a processor, when the processor executes machine readable instructions stored on a non-transitory computer readable storage medium, the processor is caused to:
perform air scans based on a predetermined reference scanning condition in a collimation condition to obtain air calibration data corresponded to the predetermined reference scanning condition; and
obtain air calibration data of non-reference scanning conditions in the collimation condition based on the air calibration data corresponded to the predetermined reference scanning condition and stored incremental differences, wherein the incremental differences stored are differences between the air calibration data of the non-reference scanning conditions and the predetermined reference scanning condition.

9. The device according to claim 8, before performing air scan based on the predetermined reference scanning condition to obtain air calibration data corresponded to the predetermined reference scanning condition, the machine readable instructions are to cause the processor to:
perform air scans based on all pre-determined scanning conditions in the collimation condition to obtain air calibration data corresponded to each of the scanning conditions;
select at least one scanning condition from all of the scanning conditions to be regarded as the reference scanning condition; and
calculate and stores the incremental differences between the reference scanning condition and the other scanning conditions in the all pre-determined scanning conditions, wherein the stored incremental differences are differences between the air calibration data of the reference scanning condition and the other scanning conditions.

10. The device according to claim 9, the machine readable instructions are further to cause the processor to:
perform air scans based on all pre-determined scanning conditions in the collimation condition to obtain air calibration data corresponded to each of the scanning conditions;
select at least one scanning condition from all of the scanning conditions to be regarded as the reference scanning condition; and
calculate and stores the incremental differences between the reference scanning condition and the other scanning conditions, wherein the stored incremental differences are differences between the air calibration data of the other scanning conditions and the reference scanning condition;
wherein each of the scanning conditions comprises at last one scan parameter of the following: a tube voltage, a rotation speed, a size of focus, a position of focus and a position of a filter.

11. The device according to claim 9, wherein in the selection of at least one reference scanning condition from all of the scanning conditions being the reference scanning condition, the machine readable instructions are further to cause the processor to:
select at least one reference scanning condition from a pre-determined ranking range of clinical usage of all scanning conditions to be the reference scanning condition.

12. The device according to claim 11, the machine readable instructions are to cause the processor to:
select the at least one reference scanning condition from a pre-determined ranking range of clinical usage of all scanning conditions to be the reference scanning condition,
wherein the pre-determined ranking range is first N scanning conditions which are sorted according to the clinical usage from highest ranking to lowest ranking in all of the scanning conditions, where N is an integer greater than or equal to 1.

13. The device according to claim 8, in the case of multiple reference scanning conditions, the obtaining air calibration data of non-reference scanning conditions in the collimation condition based on the air calibration data corresponded to the predetermined reference scanning conditions and the stored incremental differences, the machine readable instructions are to cause the processor to:
select one reference scanning condition from the multiple reference scanning conditions, wherein the selected reference scanning condition is closest to the non-reference scanning condition; and
obtain the air calibration data of non-reference scanning conditions in the collimation condition based on the air calibration data corresponded to the selected reference scanning condition.

14. The device according to claim 13, the selection of one reference scanning condition which is closest to the non-reference scanning condition from the multiple reference scanning conditions, the machine readable instructions are to cause the processor to:
select one reference scanning condition from the multiple reference scanning conditions, wherein coincidence between the scan parameters of the selected reference scanning condition and the scan parameters of the non-reference scanning condition is highest.
